(19) United States
(12) Patent Application Publication
(10) Pub. No.: US 2025/0065074 A1
(43) Pub. Date: Feb. 27, 2025

(54) BIORHYTHM IMPROVEMENT METHOD AND SYSTEM

(71) Applicant: LUPLE INC., Seoul (KR)

(72) Inventor: Yong Duck KIM, Yongin-si, Gyeonggi-do (KR)

(73) Assignee: LUPLE INC., Seoul (KR)

(21) Appl. No.: 18/724,686

(22) PCT Filed: Dec. 29, 2022
(86) PCT No.: PCT/KR2022/021649

§ 371 Date: Jun. 27, 2024

(30) Foreign Application Priority Data
Dec. 29, 2021 (KR) 10-2021-0191742
Aug. 24, 2022 (KR) 10-2022-0106391

(51) Int. Cl. A61M 21/02; A61M 21/00; G16H 50/20; G16H 70/20

(52) U.S. Cl. CPC A61M 21/02; G16H 50/20; G16H 70/20; A61M 2021/0044; A61M 2205/3303; A61M 2205/502

(57) ABSTRACT

The present invention relates to a biorhythm improvement method and system, and the method according to the present invention comprises the steps of: receiving a user biorhythm parameter; generating a user behavior guide on the basis of the user biorhythm parameter; displaying the user behavior guide on the screen of a user terminal; acquiring user behavior performance results for each user behavior item included in the user behavior guide; acquiring user sleep information; and analyzing the user behavior performance results and the user sleep information acquired during a predetermined period so as to calculate a sleep disorder correlation for each of the user behavior items. According to the present invention, a user biorhythm can be improved, and sleep disorders can be alleviated.

### TECHNICAL FIELD

The present invention relates to a biorhythm improvement method and system.

### BACKGROUND ART

About 30% of adults around the world suffer from sleep disorders. To solve the problem, most users first change their mattresses and bedding, which is not a fundamental solution. The principle of sleep is that when melatonin, the sleep hormone, increases, people fall asleep, and when the melatonin decreases, people wake up. This melatonin is controlled by the brain, and the fact that a switch for the control is light entering eyes has already been revealed through many research results. However, many users lack light during the day due to living indoors and suffer from excessive light at night due to artificial lighting, smartphones, etc. As such, the environment and lifestyle that do not match the biological rhythm are ultimately the root cause of sleep disorders. Of course, the sleep environment while sleeping is important, but activities while awake have a very large impact on sleep. Almost all species that survive on Earth have adapted to Earth's unique environment. A key element of the environment is the day/night cycle based on revolution around the sun. Therefore, a solution to reset a biological clock is absolutely necessary to restore the broken biological rhythm of modern people.

### DISCLOSURE

### Technical Problem

A technical objective to be achieved by the present disclosure is to provide a system and method capable of improving a biorhythm of a user and sleep disorders.

### Technical Solution

In an exemplary embodiment of the present invention, a biorhythm improvement method includes: receiving user biorhythm parameters; generating a user behavior guide based on the user biorhythm parameters; displaying the user behavior guide on a screen of a user terminal; acquiring user behavior performance results for each user behavior item included in the user behavior guide; acquiring user sleep information; and calculating the user sleep disorder correlation for each behavior item by analyzing user behavior performance results and user sleep information acquired during a predetermined period.

The user biorhythm parameter may include a target wake-up time and a target bedtime.

The user biorhythm parameter may further include at least one of age, gender, sunset time and sunrise time-related information, chronotype, vision correction surgery history information, eye color, sleep improvement goal, and sleep disorder factor.

The user behavior guide may include a guide for each user behavior item.

The guide for each user behavior item may include information that classifies the degree to which the corresponding user behavior is recommended differently by time zone.

The guide for each user behavior item corresponding to a combination of the user biorhythm parameters may be constructed in advance into a database.

In another exemplary embodiment of the present invention, a sleep disorder improvement method includes: receiving user biorhythm parameters; generating light therapy prescription data based on the user biorhythm parameters; outputting a notification message for a recommended use time zone of a light source so that a user uses the light source during the recommended use time zone of the light source according to light therapy prescription data; acquiring actual light source usage record data of the user; acquiring sleep record data of the user; and updating the light therapy prescription data based on the actual light source usage record data and the user sleep record data.

The light therapy prescription data may include a recommended use time zone of a light source, melanopic recommended illuminance, and a recommended amount of time to use a light source.

The light therapy prescription data corresponding to a combination of the user biorhythm parameters may be constructed in advance into a database.

The light therapy prescription data may be updated based on a sleep score corresponding to sleep quality calculated for each day during a predetermined period based on the user sleep record data and the actual light source use record data for each day.

### [Effect]

According to the present invention, it is possible to improve a biorhythm of a user and sleep disorders.

### DESCRIPTION OF DRAWINGS

FIG. 1 is a configuration diagram of a sleep improvement system according to an exemplary embodiment of the present invention.

FIGS. 2 and 3 are diagrams illustrating spectral distributions of light sources having two different wavelength bands.

FIGS. 4 and 5 illustrate a screen for receiving user sleep information according to an exemplary embodiment of the present invention.

FIG. 6 is a schematic example of the user sleep information according to an exemplary embodiment of the present invention.

FIG. 7 is a diagram for describing a user biorhythm improvement solution method according to an exemplary embodiment of the present invention.

FIG. 8 illustrates a light therapy prescription data table according to an exemplary embodiment of the present invention.

FIG. 9 is a diagram for describing a user biorhythm improvement solution method according to another exemplary embodiment of the present invention.

FIG. 10 is a diagram illustrating in detail the steps for calculating a sleep disorder correlation for each behavior item in FIG. 9.

FIG. 11 is a flowchart for describing a method of recommending a use of a lighting device according to availability of outdoor activities according to an exemplary embodiment of the present invention.

### BEST MODE

The terms are used herein for the purpose of describing the embodiments and not intended to limit the present disclosure. In the description, a singular expression also includes a plural expression unless specifically stated otherwise in the context. The terms “comprises” and/or “comprising” as used herein do not foreclose the presence or addition of one or more components other than the specified component. Throughout the description, the same reference numerals refer to the same components, and “and/or” includes each and combinations of one or more of the specified components. The terms “first”, “second”, etc. are used to describe various components, but it goes without saying that these components are not limited by these terms. These terms are only used to distinguish one component from another. Therefore, it goes without saying that a first component mentioned below may be a second component within the technical idea of the present disclosure.

A “computing device” as used herein includes all various devices capable of performing computations and providing results to a user. For example, a computing devices may include desktop PCs, notebook computers, and server computers, as well as smart phones, tablet PCs, cellular phones, PCS phones (Personal Communication Service phones), synchronous and asynchronous IMT-2000 (International Mobile Telecommunication-2000) mobile terminals, palm personal computers (Palm PCs), and personal digital assistants (PDAs).

Then, exemplary embodiments of the present invention will be described in detail with reference to the accompanying drawings so that those skilled in the art may easily practice the present invention.

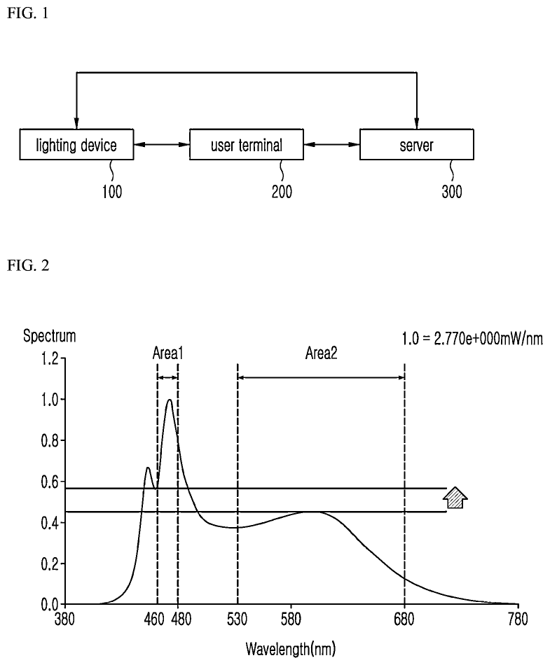

FIG. 1 is a configuration diagram of a sleep improvement system according to an exemplary embodiment of the present invention.

Referring to FIG. 1, the sleep improvement system according to the present invention may include a lighting device 100 and a user terminal 200. According to an exemplary embodiment, the sleep improvement system may further include a server 300.

The user terminal 200 and the server 300 may share and perform tasks such as data storage and data processing necessary to provide a sleep improvement digital solution according to the present invention.

The lighting device 100, the user terminal 200, and the server 300 are connected through a communication network and may exchange various information and data. A communication network may include a local area network (LAN), a metropolitan area network (MAN), a wide area network (WAN), Internet, 2G, 3G, 4G, 5G mobile communication networks, Bluetooth, Wi-Fi, Wibro, a satellite communication network, etc., and any communication method may be used, regardless of whether it is a wired or wireless communication method.

The lighting device 100 may include one or more light source elements that emit light, such as a light-emitting diode (LED) or an organic light-emitting diode (OLED).

The lighting device 100 may include a communication module (not illustrated) that supports exchanging information and data with the user terminal 200 and/or the server 300.

The lighting device 100 may be configured to generate light from a light source according to control commands or data transmitted from the user terminal 200 and/or the server 300. Of course, the lighting device 100 may be configured to emit light from a light source according to the user's manipulation. The lighting device 100 may be configured to adjust the intensity and wavelength band of light generated from the light source.

The lighting device 100 may include one or more light source elements that emit light to help reset a user's biological clock. Here, the biological clock refers to an organ responsible for the periodic rhythms, such as physiology, metabolism, and aging, of animals and plants. A specific protein is active inside the living body and acts like a kind of clock that controls a biorhythm over time, which is called a biological clock. For example, even if a user lies down all day or is in the dark, a user's body temperature changes consistently depending on the time of day and night. This fact means that a clock-like mechanism operates within the living body.

For example, the lighting device 100 may help reset the user's biological clock to day and night by using two light sources with different wavelengths. For example, a sleep improvement solution that uses a light source with a spectral distribution in a wavelength band that resets the biological clock to day for a predetermined use time (e.g., 30 minutes) in a use time zone (e.g., within 2 hours after waking up) within a predetermined time after a user wakes up and uses a light source with a spectral distribution in a wavelength band that resets the biological clock to night for a predetermined usage time (e.g., 30 minutes) within a predetermined time zone (e.g., within 1 hour before going to bed) before going to bed may be provided. Depending on individual characteristics of the user, different solutions for the use time zone and use time of the light source may be presented.

The user terminal 200 may be implemented as an information communication terminal that is equipped with a memory means and a microprocessor to have computing power, like a desktop computer, a notebook computer, a smart phone, a tablet PC, a personal digital assistant (PDA), or a web pad, wearable devices such as smart watches or augmented reality glasses, etc.

The user terminal 200 may have an application installed and executed to provide services such as sleep improvement or biorhythm improvement. The application installed on the user terminal 200 may control the operation of the lighting device 100, or collect information on the user's use of the lighting device 100 and provide the collected information to the server 300. In addition, the application installed on the user terminal 200 may receive user sleep information and provide the received user sleep information to the server 300. In addition, the application installed on the user terminal 200 may allow a user to input various types of information necessary to provide services, such as sleep improvement or biorhythm improvement, in addition to the above examples, or configure a user interface screen for display to the user and output the configured user interface screen to the screen of the user terminal 200.

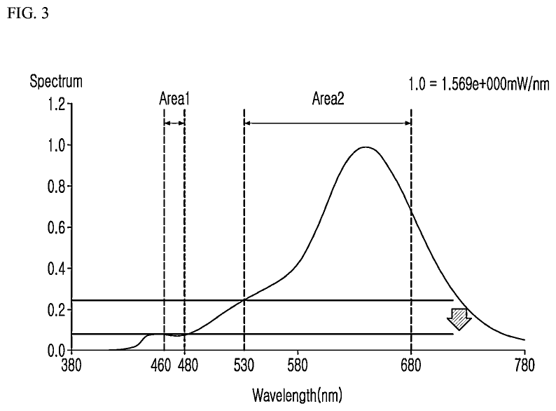

FIGS. 2 and 3 are diagrams illustrating spectral distributions of light sources having two different wavelength bands.

Referring to FIG. 2, the light source that sets the biological clock to day may be implemented as a light emitting element that generates light with a color temperature of 6500 K with a spectral distribution as illustrated in FIG. 2, that is, the increased intensity of a 460 to 480 nm wavelength band (Area 1).

Referring to FIG. 3, the light source that sets the biological clock to night may be implemented as a light emitting element that generates light with a color temperature of 1,800 K with a spectral distribution as illustrated in FIG. 3, that is, the decreased intensity of the 460 to 480 nm wavelength band (Area 1).

In order to improve sleep, the spectral distribution of the light source itself provided in the lighting device 100 is important, but the spectral distribution of light that ultimately reaches user's eyes is more important. This is because the spectral distribution may be distorted as the light from the light source passes through optical elements such as lenses and diffusers inside the lighting device 100. Therefore, it is preferable to design the lighting device 100 so that the light source has a spectral distribution as illustrated in FIGS. 2 and 3 based on the externally measured wavelength.

For example, the light source that sets the biological clock to day may be implemented using a light emitting element in which, as illustrated in FIG. 2, the light intensity in the wavelength band 460 to 480 nm area (Area 1 region), which may provide the most physiological effects, is greater than in the physiologically ineffective wavelength band 530 to 680 nm region (Area 2 region) in a visible light region. In other words, a light source with a spectral distribution in which a minimum value of the light intensity in Area 1 region is higher than a maximum value of the light intensity in Area 2 region may be used. The light source with this spectral distribution may help activate the body and improve concentration by minimizing melatonin secretion, and has an awakening effect that wakes people up.

On the other hand, as the light source that sets the biological clock to night, as illustrated in FIG. 3, a light source with a spectral distribution in which the maximum value of light intensity in the wavelength band 460 to 480 nm area (Areal region) is lower than the minimum value of the light intensity in the 530 to 680 nm region (Area 2 region) may be used. The light source with the spectral distribution may increase melatonin secretion, to help relax the body and help sleep well without interfering with rest and sleep.

Meanwhile, the light source that sets the biological clock to day may be implemented to have melanopic equivalent daylight illuminance (MEDI) of at least 200 lux at the user's eye location, and the light source that sets the biological clock to night may be implemented to have melanopic equivalent daylight illuminance (MEDI) of up to 40 lux at the user's eye location.

The melanopic equivalent daylight illuminance (MEDI) is defined as Equation

1 below.

Melanopic equivalent daylight illuminance(MEDI)=Illuminance × Melanopic daylight effective ratio(M-DER) [Equation 1]

Here, the MEDI defines the spectral power distribution of the light source light emitted from the light source of the lighting device 100 based on commission internationale de l'Eclairage (CIE) standard light D65, and the melanopic daylight effective ratio (M-DER) is defined as a ratio of relative equivalent illuminance between the light emitted from the light source and the daylight. The illuminance is the illuminance of the light source light of the lighting device 100.

For reference, the CIE standard light D65 is daylight to indicate average brightness of light during the day, and refers to a standard light source for calculating the spectral distribution of light relative to the daylight. The existing illuminance (Lux) is a measure of brightness used to quantify the effect of indoor lighting methods, but the MEDI refers to the weighted illuminance that emphasizes a reaction of eye's third intrinsically photosensitive retinal ganglion cell (ipRGC) for the physiological effect (control of melatonin secretion) described above. Therefore, the M-DER refers to the weight for the physiological effect of the comparative light source compared to the CIE standard light D65.

Since the illuminance and the melanopic equivalent daylight illuminance (MEDI) are inversely proportional to a square of a distance, in order to ensure that the light source light generated from the light source of the lighting device 100 is irradiated to the user's eyes at the targeted melanopic equivalent daylight illuminance, the lighting device 100 may include a function to measure the distance between the user's eyes and the light source of the lighting device 100. For example, the lighting device 100 includes a camera, and may recognize the eyes, nose, mouth, etc., of the user's face image captured through the camera to detect a distance between the lighting device 100 and the user or a distance between the lighting device 100 and the user's eyes. The method of calculating the distance between the camera and the user using the user's face image may be selected from various already known methods. Meanwhile, the lighting device 100 includes an ultrasonic sensor, an optical sensor (infrared ray), or the like, and may use the ultrasonic sensor, the optical sensor, etc., to detect the distance between the light source and the user's eyes. In addition to those described herein, the lighting device 100 may be implemented to detect the distance between the lighting device (or the light source of the lighting device) and the user (or the user's eyes) in various ways.

The lighting device 100 may adjust the intensity of the light source based on the distance between the lighting device (or the light source of the lighting device) and the user (or the user's eyes) to irradiate light to the user's eyes at the targeted melanopic equivalent daylight illuminance.

The lighting device 100 may provide light source use information to the user terminal 200. The light source use information may include information on the type of light source used, light source use time information, light source characteristics such as wavelength, brightness, and color temperature of the light source, etc. The type of light source used may be information divided into a light source with a spectral distribution of a wavelength band that resets the biological clock to day and a light source with a spectral distribution of a wavelength band that resets the biological clock to night. The light source use time information may include a light source use start time and end time. In addition, the light source use information may include the melanopic equivalent daylight illuminance corresponding to the user's eye location. In addition, the light source may also include light source information from external wearable devices collected through sensors that may measure melanopic equivalent daylight illuminance at the eye location.

The user terminal 200 may acquire the user sleep information. The user sleep information may be stored in the user terminal 200 and/or stored in the server 300.

The user sleep information includes at least actual falling asleep time, sleep segment information, and actual time of waking up from sleep. The sleep segment information may include the number of times of sleep segments and sleep segment time information. The number of times of sleep segments refers to the number of times people wake up during sleep. The sleep segment time information refers to information on the time it takes to wake up during sleep and the time it takes to fall asleep again. For example, when people wake up twice during sleep, the number of times of sleep segments is twice, and the sleep segment time information includes information on the time people wake up for the first time and the time it takes them to fall asleep again and information on the time people wake up for the second time and the time it takes them to fall asleep again. Meanwhile, the user sleep information may also include information on the user's subjective evaluation of the sleep quality.

The user terminal 200 may manually receive the user sleep information from the user through the sleep information input screen. That is, the user may write his or her sleep diary by manually inputting the sleep information through the sleep information input screen. Of course, according to an exemplary embodiment, a separate sleep measuring device (not illustrated) (e.g., a wearable device such as a smart watch worn by the user or a device capable of measuring other user sleep information) can be implemented to detect information corresponding to the user sleep, and provide the detected information to the user terminal 200 in a wired or wireless manner.

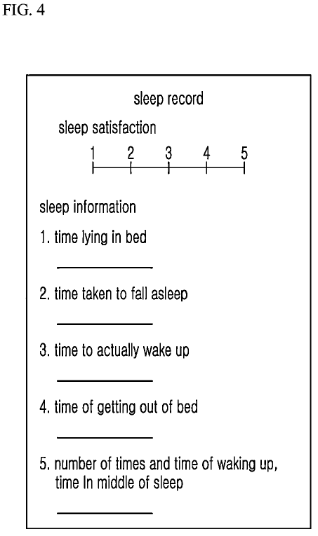

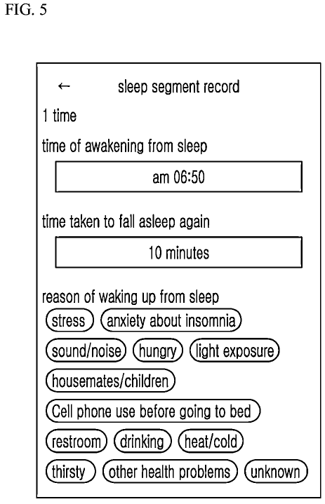

FIGS. 4 and 5 illustrate a screen for receiving user sleep information according to an exemplary embodiment of the present invention.

Referring to FIG. 4, a user evaluation of last night's sleep quality, that is, sleep satisfaction, may be received according to a predetermined rating. For example, as illustrated in FIG. 4, the sleep satisfaction may be received from the user on a 1 to 5 point scale. Of course, the score scale for evaluating sleep satisfaction may vary depending on the exemplary embodiment. In addition, rather than directly presenting the score corresponding to sleep satisfaction, it is also possible to present the sleep satisfaction as very bad, bad, normal, good, very good, etc., and convert this into a set score.

In addition, as illustrated in FIG. 4, 1) the time of lying in bed, 2) the time taken to fall asleep after lying in bed (sleep latency), 3) the actual time of opening eyes (time of waking up from sleep), 3) the time of getting out of bed (wake-up time), 4) the number of times and time of waking up during sleep (number of times of sleep segments and sleep segment time).

Meanwhile, according to the exemplary embodiment, when the number of times waking up during sleep is input, as illustrated in FIG. 5, a predetermined reason for sleep segment may be presented and may be selected by the user. Reasons for sleep segment may include stress, anxiety about insomnia, sound/noise, hunger, light exposure, heat/cold, etc. Of course, other reasons for sleep segments other than those illustrated here may be presented and selected.

In general, it is difficult for people to know their sleep time directly. However, it is possible to roughly know the time of lying in bed to fall asleep and the time it takes to fall asleep from that time. Therefore, the sleep time may be obtained by adding ‘2) the time it takes to fall asleep after lying in bed (sleep latency)’ from ‘1) the time of lying in bed.’

Likewise, when there is the sleep segment in which people wake up in the middle, as illustrated in FIG. 5, when the time of waking up from sleep and the time it takes to fall back to sleep (sleep segment time) are received, the time it takes to fall asleep again after the sleep segment may be obtained.

Instead of receiving the actual time of opening eyes input from the user, according to the exemplary embodiment, it is possible to obtain the actual time of opening eyes (time of waking up from sleep) by receiving the time of getting out of bed and the time of lying in bed after waking up and then calculating reversely.

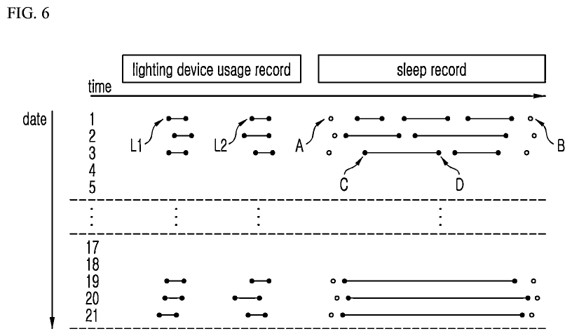

FIG. 6 is a schematic example of the user sleep information according to an exemplary embodiment of the present invention.

Referring to FIG. 6, the horizontal axis represents the time of the day from 0:00 to 24:00, and the vertical axis represents the date. L1 represents the use information of the light source that resets the biological clock to day, which is an example of the use information of the light source as a line connecting the use start time of the light source and the use end time. Likewise, L2 represents the use information of the light source that resets the biological clock to night. Meanwhile, the use time of the light source may be recognized by the length of L1 and L2, but the light source can be displayed in color to be more intuitively recognized. For example, depending on the type of light source, the light source that resets the biological clock to day and the light source that resets the biological clock to night may be divided in color, and when the light source is used sufficiently for more than the recommended time, the light source can be displayed in blue, when the light source is used within the recommended time range, the light source can be displayed in green, when the light source is used less than the recommended time, the light source can be indicated in red, or the like. L1 and L2 may not be displayed when not used, and may be written multiple times when used multiple times.

In FIG. 6, A represents the time of lying in bed, B represents the time of getting out of bed, C represents sleep time, and D represents the time of waking up from sleep. As described above, sleep records may be displayed based on records written by the user in a sleep diary. Of course, it is also possible to display sleep records based on the sleep information detected from the sleep measuring device. In cases where the sleep stage may be obtained from the sleep measuring device, deep sleep and light sleep may be displayed in color. In addition, it is possible to display the sleep record based on the user's sleep diary and the sleep record based on the sleep information detected from the sleep measuring device together on one screen. It may help a doctor or an administrator diagnose the patient's condition by looking at both the user's subjective sleep record and the objective sleep record by the sleep measuring device.

Looking at the sleep record in FIG. 6, the user initially showed the sleep segment in which the user woke up 2 to 3 times during sleep, but by continuously using the lighting device, it is possible to confirm that the user was sleeping soundly without the sleep segment. In this way, by looking at the lighting device use record (light source use information) and sleep record (user sleep information) together illustrated in FIG. 6, the user, doctor, or administrator may see at a glance how the sleep quality of the user is improved.

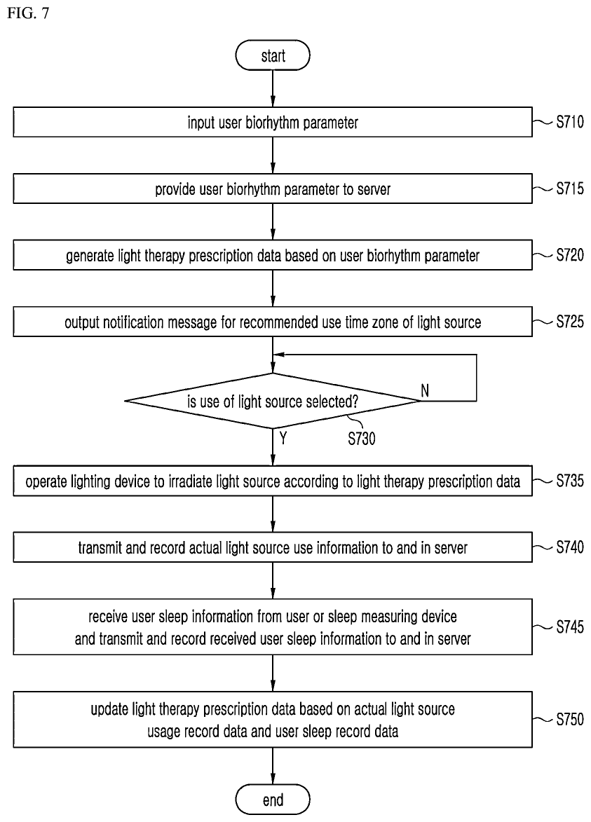

FIG. 7 is a diagram for describing a user biorhythm improvement solution method according to an exemplary embodiment of the present invention.

Referring to FIG. 7, first, the user terminal 200 may receive user biorhythm parameters (S710). The user biorhythm parameters input in step S710 include a target wake-up time and a target bedtime. In addition, in step S710, the user biorhythm parameters may also include additional information such as age, gender, sunset time and sunrise time-related information, chronotype, vision correction surgery history information, eye color, sleep improvement goal, or sleep disorder factor.

The age may be implemented by presenting an age range, selecting from among them, or by inputting the actual user age.

The sunset time and sunrise time-related information is information that may be used to obtain the sunset time and sunrise time at the user's current location. For example, the user's current location information may be used as the sunset time and sunrise time-related information. When the user's current location is known, it may be confirmed the sunset time and sunrise time for that location. The sunset time and sunrise time-related information may be information selecting the area where the user lives, or may be GPS location information acquired from a GPS reception module of the user terminal 200.

The chronotype is an index that divides the tendency of the time zone in which a person is most actively awake and asleep during the day according to a circadian rhythm, and may be divided into a morning type and an evening type, or a morning type, an intermediate type, and an evening type, and may be presented in more detail than the example and selected by the user.

The vision correction surgery history information may present LASIK or LASEK surgery and may be selected by the user, and the eye color may also present a predetermined color and may be selected by the user.

The sleep improvement goal or the sleep disorder factor may present the predetermined type and may also be selected by the user. For example, it is possible to present and select the type of sleep improvement goal, such as wanting to fall asleep easily, not wanting to wake up in the middle, not wanting to wake up too early, feeling refreshed when waking up, etc. Alternatively, it is possible to present and select the type of sleep disorders such as taking a long time to fall asleep, waking up frequently while sleeping, not feeling refreshed after sleeping, and waking up too early.

Next, the user terminal 200 may provide the user biorhythm parameters input in step S710 to the server 300 (S715).

Thereafter, the server 300 may generate the light therapy prescription data based on the user biorhythm parameters (S720). The light therapy prescription data may include the recommended use time zone of the light source, the melanopic recommended illuminance, and the recommended amount of time to use the light source. To this end, the server 300 may build and store the light therapy prescription data corresponding to the combination of the biorhythm parameters in the database in advance. Here, using the light source means that the user exposes the light emitted from the light source of the lighting device 100.

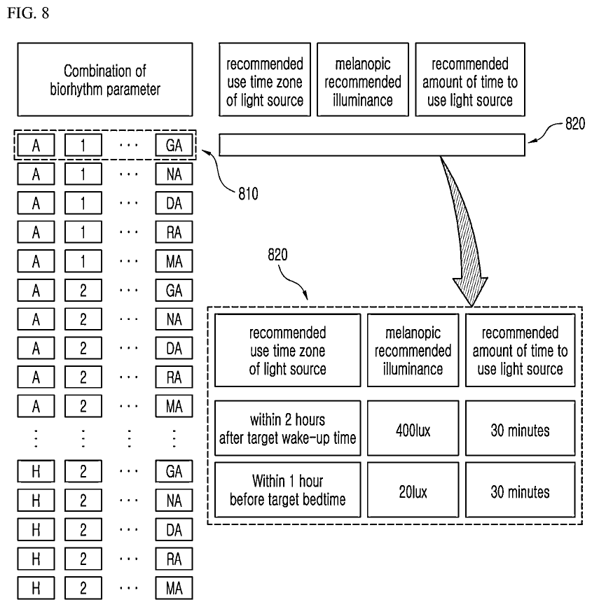

FIG. 8 illustrates a light therapy prescription data table according to an exemplary embodiment of the present invention.

Referring to FIG. 8, regarding a biorhythm parameter combination 810 composed of ‘A, 1, . . . , GA,’ light therapy prescription data 820 may be mapped in advance and stored on the server 300. The light therapy prescription data may be mapped in advance for each biorhythm parameter combination.

The recommended use time zone of the light source may be predetermined as illustrated in FIG. 8 based on the target wake-up time and target bedtime. For example, it may be predetermined, such as ‘within 2 hours after the target wake-up time’, ‘within 1 hour before the target bedtime’, etc. Of course, according to the exemplary embodiment, it may be set to a specific time zone, such as AM 10:00 to 12:00.

The melanopic recommended illuminance and recommended amount of time to use the light source may also be predetermined. In the case of the melanopic recommended illuminance and light source use time amount, the melanopic equivalent daylight illuminance increases or decreases from the default values depending on age based on the default values such as ‘400 lux, 30 minutes’, and ‘20 lux, 30 minutes’, and when the light blurring is severe due to LASIK or LASEK surgery, the light therapy prescription data may be set in advance according to the characteristics of the biorhythm parameter combination by lowering the melanopic equivalent daylight illuminance, increasing the use time, etc.

Of course, according to the exemplary embodiment, a training data set to which the combination of the user biorhythm parameters and the light therapy prescription data are mapped may be constructed, and a neural network model may be trained with the constructed training data set. In this case, a data pair of the combination of the user biorhythm parameters and the light therapy prescription data, which have previously been proven to improve sleep, may be constructed as the training data in advance. The trained neural network model may output the light therapy prescription data when the user biorhythm parameters are input. The neural network model may be in the form of a machine learning algorithm such as a convolutional neural network (CNN). The convolutional neural networks may also be implemented as deep CNN models such as AlexNet, VGGNet, GoogLeNet, and ResNet. According to the exemplary embodiment, a light therapy prescription algorithm can be derived by the regression analysis of the data set to which the combination of the user biorhythm parameters and the light therapy prescription data are mapped.

Next, the user terminal 200 may output a notification message for the recommended use time zone of the light source so that the user may use the light source during the recommended use time zone of the light source according to the light therapy prescription data generated by the server 300 (S725). For example, the notification message for the recommended use time zone of the light source may be output at the start time of the recommended use time zone of the light source or before a certain time from the start time. The notification message for the recommended use time zone of the light source may be continuously displayed on the screen of the user terminal 200 until the user uses the lighting device 100. Of course, it is also possible to confirm the recommended use time zone of the light source and then output the notification message for the recommended use time zone of the light source at the time set by the user. Meanwhile, the notification message for the recommended use time zone of the light source may include not only the recommended use time zone of the light source, but also the melanopic recommended illuminance and/or the recommended amount of time to use the light source according to the exemplary embodiment.

Thereafter, when the user confirms the notification message for the recommended use time zone of the light source and selects the use of the light source in the user terminal 200 (S730-Y), the user terminal 200 may operate the lighting device 100 to irradiate the light source for the predetermined recommended amount of time to use the light source with the predetermined melanopic recommended illuminance according to the light therapy prescription data (S735). According to the exemplary embodiment, the user may confirm the notification message for the recommended use time zone of the light source in step S725 and then manually operate the lighting device 100 according to the light therapy prescription data.

Meanwhile, according to the exemplary embodiment, the lighting device 100 may be implemented to receive the light therapy prescription data from the user terminal 200 or the server 300, and to automatically irradiate light with the predetermined melanopic recommended illuminance for the recommended amount of time to use the light source when the recommended use time zone of the light source is reached. In this case, the lighting device 100 may be implemented to irradiate the light source when the user is located within a predetermined range from the lighting device 100, when there are no obstacles between the light source and the eyes while the user is located within the predetermined range, or when the user is wearing the wearable light therapy device. When the distance between the light source and the user's eyes is known, it is possible to operate so that the melanopic equivalent daylight illuminance reaching the eyes is kept constant according to the distance calculation.

The lighting device 100 may transmit and record the actual light source use information to and in the server 300 (S740). In step S740, the actual light source use information may be transmitted to the server 300 through the user terminal 200. The actual light source use information is information on the user's actual use of the lighting device, and may include an actual light source use start time, an actual light source use end time, actual melanopic equivalent daylight illuminance, and an actual light source use time amount, etc.

According to one exemplary embodiment, it can be implemented by receiving the actual light source use information from the user in the user terminal 200. In this case, just as the user writes the sleep record above, the actual light source use record may be implemented to be input by the user from the user terminal 200.

Meanwhile, the user terminal 200 may receive the user sleep information from the user or the sleep measuring device and transmit and record the received user sleep information to and in the server 300 (S745). The user sleep information records are the same as described above.

The server 300 may update the light therapy prescription data based on the actual light source usage record data and the user sleep record data collected during a predetermined period for the user (S750). For example, the sleep score for the sleep quality for each day during a predetermined period may be calculated using the user sleep record data. Based on the light therapy prescription data generated in step (S720), the actual light source usage record data and the user sleep record data collected by repeating steps (S725) to (S740) during a predetermined period may be used to update the light therapy prescription data. Most simply, the light therapy prescription data may be updated with the light source use time and the melanopic equivalent daylight illuminance on the day when the sleep score was the best. Of course, the neural network model is trained using the training data composed of the light source use time, the melanopic equivalent daylight illuminance, and the sleep score, and can be implemented to output the optimal light therapy prescription data by receiving the actual light source usage record data and the user sleep record data collected during a certain period. The sleep score calculation method is described in detail below.

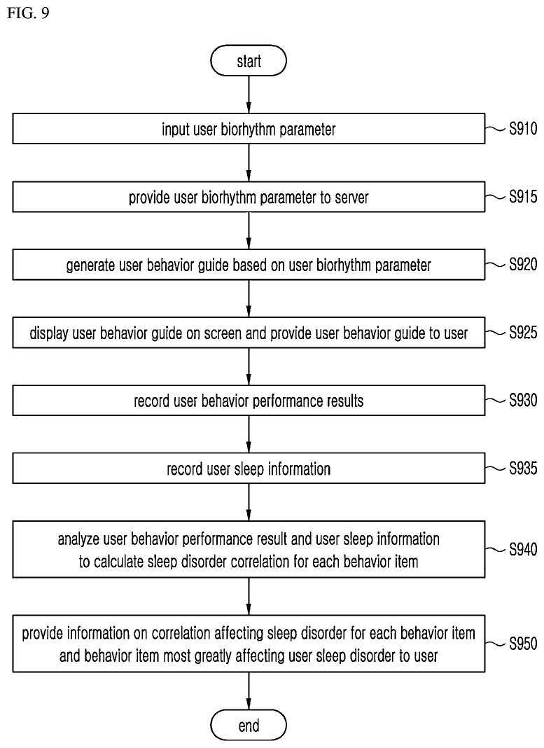

FIG. 9 is a diagram for describing a user biorhythm improvement solution method according to another exemplary embodiment of the present invention.

Referring to FIG. 9, first, the user terminal 200 may receive the user biorhythm parameters (S910). The user biorhythm parameters input in step S910 include the target wake-up time and the target bedtime. In addition, in step S910, the user biorhythm parameters may also include additional information such as age, gender, sunset time and sunrise time-related information, chronotype, vision correction surgery history information, eye color, sleep improvement goal, or sleep disorder factor.

Next, the user terminal 200 may provide the user biorhythm parameters input in step S910 to the server 300 (S915).

Thereafter, the server 300 may generate the user behavior guide based on the user biorhythm parameters (S920).

The user behavior guide (guide) may include a guide for each behavior item. The guide for each behavior item may be, for example, a sunlight viewing guide, a caffeine guide (or drug guide), a meal guide, an exercise guide, etc. According to the exemplary embodiment, the behavior items included in the user behavior guide may vary. Here, the case where the user behavior guide includes the sunlight viewing guide, the caffeine guide, the meal guide, and the exercise guide will be described.

The guide for each behavior item may include information that classifies the degree to which the user behavior corresponding to the item is recommended differently by time zone. For example, depending on the degree of the recommendation, the time zone may be divided into a highly recommended time zone, a recommended time zone, a normal time zone, a warning time zone, a prohibited time zone, etc.

The highly recommended time zone and the recommended time zone are time zones in which the behavior has a positive effect on sleep and are recommended time zones for the behavior. The highly recommended time zone is a time zone in which the behavior has a more positive effect on sleep than the recommended time zone. On the other hand, the warning time zone and the prohibited time zone are time zones in which the behavior has a negative effect on sleep and are time zones where the behavior is not recommended or prohibited. The prohibited time zone is a time zone in which the behavior is not more actively recommended than the warning time zone. In addition to the example here, the time zone may be further or less subdivided.

For example, in the case of the sunlight viewing guide, the highly recommended time zone is basically 4 hours after waking up, but may be set differently from 2 to 6 hours depending on the chronotype or the type of sleep disorder. In general, it is common to view sunlight for about 30 minutes 14 to 15 hours before the target bedtime, but when a user who usually sleeps late wants to go to bed early, since the time to view the first sunlight should be earlier than the existing sunlight viewing time, the highly recommended time zone may be set to 2 hours after waking up to view sunlight within 2 hours of waking up. In addition, users who wake up an hour before sunset due to shift work where day and night are reversed may set the highly recommended time zone to ‘after waking up and before sunset’ when they may view at least a little bit of the sun after waking up. Since there are various cases depending on the type of user, the time zone information set as the sunlight viewing guide according to the combination of user biorhythm parameters may be constructed and stored in a database in advance and used.

Likewise, the caffeine guide, the meal guide, and the exercise guide may be used by constructing and storing the preset time zone information in a database according to the combination of the user biorhythm parameters. Meanwhile, in the case of the caffeine intake time, considering the relationship between the caffeine and sleep, it is better not to consume the caffeine, so there is no need for the highly recommended time zone, the recommended time zone, the normal time zone, etc., and only the warning time zones and prohibited time zones may be constructed and stored in the database according to the combination of user biorhythm parameters and used. The warning time zone and prohibited time zone may be set in advance according to the combination of the user biorhythm parameters based on the target bedtime, taking into account the caffeine half-time (generally about 6 to 8 hours).

Meanwhile, the guide for each behavior item may additionally include information on the number of times of recommendations or the amount of recommended time for the corresponding behavior. For example, the sunlight viewing guide may also include information such as viewing sunlight twice and viewing sunlight for 30 minutes each time. The exercise guide may also include information on the number of times of exercises and the recommended amount of time per exercise, and may also include information on exercise intensity. On the other hand, the caffeine guide may include information on the number of times of caffeine intake limits, for example, 2 cups of coffee (100 mg of caffeine or less) or less.

Next, the user terminal 200 may display the user behavior guide provided from the server 300 on the screen and provide the displayed user behavior guide to the user (S925). The user behavior guide may be displayed in the form of text or provided as a diagram to make it easier for users to understand. For each behavior item, the highly recommended time zone, the recommended time zone, the normal time zone, the warning time zone, and the prohibited time zone are displayed in a visually divided graph form, allowing users to intuitively divide and recognize the time zones according to the user behavior guide.

Thereafter, the user terminal 200 may receive the user behavior performance results and transmit and record the received user behavior performance results to and in the server 300 (S930). Users may input behavior results for each behavior item included in the user behavior guide. Most simply, the default values may be input by clicking an input button for each behavior item displayed on the screen. For example, when the input button corresponding to viewing sunlight is clicked once, it may be processed as if the user viewed sunlight for a predetermined amount of time, for example, 30 minutes at the time of clicking. Likewise, when the button corresponding to the exercise item is clicked once, it may be processed as if the user performed exercise for a predetermined amount of time, for example, 30 minutes at the time of clicking. Meanwhile, when the button corresponding to the meal or caffeine item is clicked once, it may be processed as if the user ate a meal or drank coffee once at the time of the clicking.

Of course, according to the exemplary embodiment, it may be implemented so that the user manually inputs the user behavior performance results. For example, in the case of the sunlight viewing item, it may be implemented so that the user directly inputs the time when he or she started and finished viewing sunlight. In the case of the caffeine item, it is also possible to allow the user to input the time the coffee was drunk and the number of cups of coffee. In the case of the exercise or meal item, it is also possible to allow the user to directly input exercise time, meal time, exercise intensity, etc.

Meanwhile, it is also possible to automatically process the step (S930) of inputting the user behavior performance results. For example, when the location recognition function of the user terminal 200 recognizes that the user has visited a specific place, such as a restaurant, a coffee shop, or a gym, it is automatically processed as if the user ate, drank coffee, or exercised at that time. In addition, when the user is recognized as having engaged in activities such as taking a walk outdoors during the day, it may be automatically processed as having viewed sunlight at that time. In addition, it may be processed as if the user ate a meal or drank coffee based on user's credit card payment information. In addition to the example here, it is possible to automatically process step (S930) by adopting a technical method that may recognize the user behavior performance results.

Meanwhile, the user terminal 200 may receive the user sleep information and transmit and record the received user sleep information to and in the server 300 (S935). The user sleep information may include the user evaluation of sleep quality, the sleep latency, the sleep time, the number of times of sleep segments, the sleep disorder factors, etc. The user sleep information can be received manually from the user, or can be input automatically by linking with the sleep measuring device. Alternatively, it is possible to simultaneously perform the manual and automatic input of the user sleep information. The user sleep information records are the same as described above.

Thereafter, the server 300 may calculate each sleep disorder correlation for each behavior item by analyzing the user behavior performance results and the user sleep information collected during a predetermined period (S940).

Step 940 will be described in more detail.

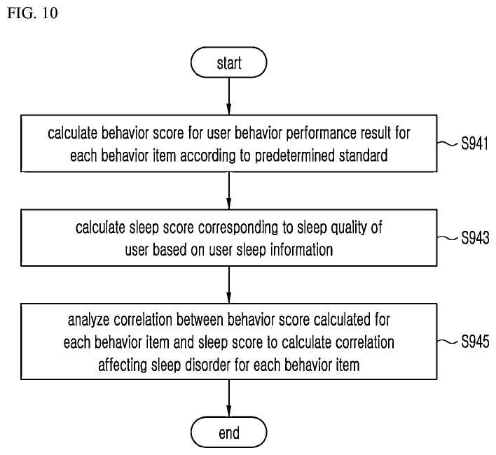

FIG. 10 is a diagram illustrating in detail the steps for calculating a sleep disorder correlation for each behavior item in FIG. 9.

Referring to FIG. 10, the server 300 may calculate the behavior scores for the user behavior performance results for each behavior item according to a predetermined standard (S941).

For example, in the case of the sunlight viewing item, the behavior scores may be calculated as follows.

TABLE 1

| | Highly | Recom- | | Warn- | Prohib- |
|---|---|---|---|---|---|
| Division | recommended | mended | Normal | ing | ited |
| One time | 3 | 2 | 1 | 0 | 0 |
| Twice or more | 4 | 2.5 | 1.3 | 0 | 0 |

First, as illustrated in Table 1, scores granted according to the sunlight viewing frequency may be set in advance for the highly recommended time zone, the recommended time zone, the normal time zone, the warning time zone, the prohibited time zone, etc. For example, when the user views sunlight twice or more in the highly recommended time zone and views sunlight once in the recommended time zone, a total of 6 points (4+2) may be granted. Only if the user views sunlight for more than 30 minutes, viewing sunlight may be counted as one time. If necessary, the sunlight viewing behavior score may be calculated by multiplying the score granted to the number of times of behaviors per time zone by the weight.

TABLE 2

| Age | Weight |
|---|---|
| to 25 | 1.2 |
| 26 to 65 | 1 |
| 65 to | 0.8 |

Table 2 shows an example of sunlight viewing score weights according to age. In general, the older the user get, the more sunlight reaches the retina. Reflecting this, the sunlight viewing behavior score may be calculated by multiplying the score granted in Table 1 by the weight. Of course, the vision correction surgery history information, the eye color, etc., may be reflected in the weight. According to the exemplary embodiment, it is also possible to limit the maximum or minimum score range for each behavior item. The score granted to the number of times of behaviors per time zone is 6 points, and for a user aged 27 years, 6 points are calculated. However, when the maximum score is limited to 5 points, the final sunlight viewing behavior score may be granted as 5 points.

Meanwhile, in the case of the caffeine, exercise, and meal items, excluding viewing sunlight, it is also possible to grant scores according to behavior by time zone, as shown in Table 3.

TABLE 3

| | Highly | | | Warn- | Prohib- |
|---|---|---|---|---|---|
| Division | recommended | Recommend | Normal | ing | ited |
| Caffeine | 0 | 0 | 0 | −0.5 | −1 |
| Exercise | 0 | 0.5 | 0 | −0.5 | −1 |
| Meal | 0 | 0 | 0 | −0.5 | −1 |

In the case of the caffeine or meal, only the warning time zone and prohibited time zone may be included in the behavior guide, and a negative score may be granted when the behavior is performed in the warning or the prohibited time zone. In the case of the exercise, it is possible to grant positive points because the exercise in the recommended time zone helps with sleep, and grant negative points to the warning time zones and the prohibited time zone. According to the exemplary embodiment, it is possible to additionally accumulate and grant the corresponding points for each number of behaviors, or to grant the corresponding points only depending on whether or not the behavior occurred, regardless of the number of times of behaviors in the corresponding time zone. The example described above is only an example of granting behavior scores, and the standards for granting the behavior scores may vary according to the exemplary embodiment.

Then, the sleep score corresponding to the user's sleep quality is calculated based on the user sleep information (S943). The sleep score may be calculated using a predetermined algorithm using factors such as the sleep time, the difference between the target sleep viewpoint and the actual sleep viewpoint, the number of times of sleep segments, the number of sleep disorder factors, and the difference between the target sleep time and the actual sleep time. Meanwhile, according to the exemplary embodiment, the sleep score may include the evaluation of the user's subjective sleep quality.

Hereinafter, an example of a specific method for calculating a sleep score will be described.

First, the recommended sleep time for each age group may be set in advance, as shown in Table 4 below.

TABLE 4

| Age | Min | Max |
|---|---|---|
| to 5 | 10 | 14 |
| 6 to 13 | 9 | 11 |
| 14 to 18 | 8 | 10 |
| 19 to 25 | 7 | 9 |
| 26 to 64 | 7 | 9 |
| 65 to | 7 | 8 |

As shown in Table 5 below, a score standard for the time taken to fall asleep (sleep latency) may be prepared in advance.

TABLE 5

| | to 15 | 15 to 30 | 30 minutes to 1 | 1 hour or |
|---|---|---|---|---|
| Division | minutes | minutes | hour | more |
| Score | 0 | −0.1 | −0.2 | −0.3 |

In addition, as shown in Table 6 below, a score standard for the difference between the actual sleep time (excluding sleep segment time) and the recommended sleep time may be prepared in advance. When the actual sleep time is within the Min to Max range of the recommended sleep time, 0.3 points may be granted, and when the actual sleep time is outside the Min to Max range, −0.2 or −0.5 points may be granted.

TABLE 6

| Between Min to Max | Recommended sleep time | 0.3 |
|---|---|---|
| Min or less/Max or more | Within ±1 hour | −0.2 |
| Min or less/Max or more | ±2 hours or more | −0.5 |

In addition, a score standard for the number of times of sleep segments may be prepared in advance, as shown in Table 7 below.

TABLE 7

| No | Once | Twice | Three times or more |
|---|---|---|---|
| 0.2 | 0 | −0.1 | −0.3 |

As shown in Table 8 below, a score standard for the number of reasons for sleep segments (number of sleep disorder factors) may be prepared in advance.

TABLE 8

| No | One | Two | Three or more |
|---|---|---|---|
| 0.1 | 0 | −0.1 | −0.2 |

The sleep score may be calculated by adding up the corresponding scores according to the score granting standard set in Tables 5 to 8. The example described above is only an example of granting sleep scores, and the standards for granting the behavior scores may vary according to the exemplary embodiment.

Next, the server 300 analyzes the correlation between the behavior score and sleep score calculated for each behavior item to calculate the correlation that affects sleep disorders for each behavior item, and may also determine the behavior item that affects the user's sleep disorder the most (S945). For example, it is possible to determine the correlation by obtaining the correlation coefficient between the behavior score and sleep score calculated for each behavior item. For example, the Pearson correlation coefficient between each behavior item and sleep may be obtained based on the behavior score and sleep score for each behavior item during a predetermined period.

Referring back to FIG. 9, next, the user terminal 200 may provide the user with the information on the correlation affecting the sleep disorder for each behavior item previously obtained from the server 300 and the behavior item that affects on the user's sleep disorder the most (S950).

Meanwhile, the user terminal 200 may provide data on how much the user behaved according to the given behavior guide.

The server 300 may update the user behavior guide based on the user behavior result records and user sleep record data collected during a predetermined period for the corresponding user. For example, the sleep score for the sleep quality for each day during a predetermined period may be calculated using the user sleep record data. Most simply, the user behavior guide may be updated to respond to the user behavior results on the days with the best sleep scores. Of course, the neural network model is trained using the training data composed of the user behavior results and the sleep scores, and can be implemented to output the optimal user behavior guide by receiving the user behavior result records and the user sleep records collected for the user over a certain period of time.

Meanwhile, in the case of sunlight viewing, the user terminal 200 can also support the user to achieve his/her behavioral goal by linking with the lighting device 100. For example, when the therapeutic lighting device 100 is registered in the user terminal 200, the lighting device 100 may be used in the case where sunlight viewing is inappropriate during outdoor activities, the case where the user's sleep time is reversed between day and night, or the like to suggest to the user to perform the sunlight viewing.

The case where the sunlight viewing is inappropriate during outdoor activities may include the case where it rains or snows, the case where ultraviolet rays are strong, the case where the concentration of fine dust is high, the case where the temperature is too high or too low, etc.

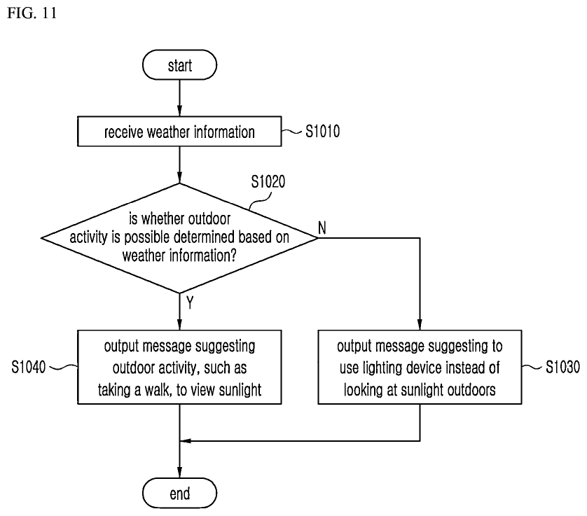

FIG. 11 is a flowchart for describing a method of recommending a use of a lighting device according to availability of outdoor activities according to an exemplary embodiment of the present invention.

Referring to FIG. 11, first, the user terminal 100 may receive weather information from a weather server or the server 300 that provides weather information (S1010). The weather information may include weather items such as temperature, precipitation, rainfall, fine dust concentration, and ultraviolet ray concentration.

Next, the user terminal 100 may determine whether outdoor activities are possible based on the weather information (S1020).

When one or more weather items included in the weather information exceed predetermined standards, it is determined that outdoor activities to view sunlight are impossible (S1020-N), and a message suggesting using the lighting device 100 instead of viewing sunlight outdoors may be output (S1030).

Meanwhile, when all the weather items included in the weather information satisfy predetermined standards, the user terminal 100 may determine that outdoor activities to view sunlight are possible (S1020-Y), and output a message suggesting outdoor activities, such as taking a walk to view sunlight (S1040).

So far, the operations of the user terminal 200 and the server 300 have been described separately, but according to the exemplary embodiment, it is also possible to implement all the functions in the user terminal 200.

The embodiments described above may be implemented as a hardware component, a software component, and/or a combination of a hardware component and a software component. For example, the devices, methods, and components described in the embodiments may be implemented by using one or more general computing device or specific-purpose computing device such as a processor, a controller, an arithmetic logic unit (ALU), a digital signal processor, a microcomputer, a field programmable gate array (FPGA), a programmable logic unit (PLU), a microprocessor, or any other device capable of executing instructions and responding thereto. The processing device may execute an operating system (OS) and one or more software applications executed on the operating system. Further, the processing device may access, store, operate, process, and generate data in response to the execution of software. For convenience of understanding, it is described in certain examples that one processing device is used, but one of ordinary skill in the art may understand that the processing device may include a plurality of processing elements and/or a plurality of types of processing elements. For example, the processing device may include a plurality of processors or one processor and one controller. In addition, other processing configurations such as a parallel processor are possible.

The software may include a computer program, code, instructions, or a combination of one or more of the above, and may configure the processing unit, or instruct the processing unit independently or collectively to operate as desired. Software and/or data may be interpreted by the processing device or, in order to provide instructions or data to the processing device, may be embodied in any type of machine, component, physical device, virtual equipment, computer storage medium or device, or signal wave transmission, permanently or temporarily. The software may be distributed over networked computer systems and stored or executed in a distributed manner. The software and data may be stored on one or more computer-readable recording media.

The method according to the embodiment may be implemented in the form of program instructions that can be executed through various computer means and recorded in a computer-readable medium. The computer readable medium may include program instructions, data files, data structures, and the like alone or in combination. The program instructions recorded on the medium may be those specially designed and configured for the purposes of the embodiments, or may be known and available to those skilled in computer software. Examples of computer readable recording medium include magnetic media such as hard disks, floppy disks, and magnetic tape, optical media such as CD-ROMs and DVDs, magneto-optical media such as floptical disks, and hardware devices specifically configured to store and execute program instructions such as ROM, RAM, flash memory, and the like. Examples of the program instructions include machine language codes such as those generated by a compiler, as well as high-level language codes that may be executed by a computer using an interpreter, and so on. The hardware device described above may be configured to operate as one or more software modules in order to perform the operations according to the embodiments, and vice versa.

As described above, although the embodiments have been described with reference to the limited drawings, a person of ordinary skill in the art can apply various technical modifications and variations based on the above. For example, even when the described techniques are performed in the order different from the method described above, and/or even when the components of the system, structure, device, circuit, and the like are coupled or combined in a form different from the way described above, or replaced or substituted by other components or equivalents, an appropriate result can be achieved.

1. A biorhythm improvement method, comprising:
receiving user biorhythm parameters;
generating a user behavior guide based on the user biorhythm parameters;
displaying the user behavior guide on a screen of a user terminal;
acquiring user behavior performance results for each user behavior item included in the user behavior guide;
acquiring user sleep information; and
calculating a user sleep disorder correlation for each behavior item by analyzing the user behavior performance results and the user sleep information acquired during a predetermined period.

2. The biorhythm improvement method of claim 1, wherein the user biorhythm parameter includes a target wake-up time and a target bedtime, and
the user biorhythm parameter further includes at least one of age, gender, sunset time and sunrise time-related information, chronotype, vision correction surgery history information, eye color, sleep improvement goal, and sleep disorder factor.

3. The biorhythm improvement method of claim 2, wherein the user behavior guide includes a guide for each user behavior item, and
the guide for each behavior item includes information that classifies a degree to which a corresponding user behavior is recommended differently by time zone.

4. The biorhythm improvement method of claim 3, wherein the guide for each user behavior item corresponding to a combination of the user biorhythm parameters is constructed in advance into a database.

5. A sleep disorder improvement method, comprising:
receiving user biorhythm parameters;
generating light therapy prescription data based on the user biorhythm parameters;
outputting a notification message for a recommended use time zone of a light source so that a user uses the light source during the recommended use time zone of the light source according to light therapy prescription data;
acquiring actual light source usage record data of the user;
acquiring sleep record data of the user; and
updating the light therapy prescription data based on the actual light source usage record data and the user sleep record data.

6. The sleep disorder improvement method of claim 5, wherein the user biorhythm parameter includes a target wake-up time and a target bedtime, and
the user biorhythm parameter further includes at least one of age, gender, sunset time and sunrise time-related information, chronotype, vision correction surgery history information, eye color, sleep improvement goal, and sleep disorder factor.

7. The sleep disorder improvement method of claim 6, wherein the light therapy prescription data includes the recommended use time zone of the light source, melanopic recommended illuminance, and a recommended amount of time to use the light source.

8. The sleep disorder improvement method of claim 7, wherein the light therapy prescription data corresponding to a combination of the user biorhythm parameters is constructed in advance into a database.

9. The sleep disorder improvement method of claim 7, wherein the light therapy prescription data is updated based on a sleep score corresponding to sleep quality calculated for each day during a predetermined period and the actual light source usage record data for each day.
